(19) United States
(12) Patent Application Publication
(10) Pub. No.: US 2011/0245775 A1
(43) Pub. Date: Oct. 6, 2011

(54) TAPERED SHEATH

(75) Inventor: Kurt J. Tekulve, Ellettsville, IN (US)

(73) Assignee: Cook Incorporated, Bloomington, IN (US)

(21) Appl. No.: 12/752,559

(22) Filed: Apr. 1, 2010

(51) Int. Cl. A61M 25/01; B29C 41/30

(52) U.S. Cl. 604/171; 604/526; 264/171.12

(57) ABSTRACT

A sheath and a method for making said sheath are provided. The sheath includes with an inner liner defining a passageway about a longitudinal axis extending longitudinally therethrough. A coil is fitted around at least a part of the inner liner. The coil has a series of windings that are locatable at a continuously smaller distance from the longitudinal axis to form a taper in a distal direction. The tube further includes an outer layer positioned longitudinally over the coil that is adapted to adhere to the inner liner. The coil may be spirally wound such that the windings are disposed at a continuously smaller distance from the longitudinal axis in a longitudinal direction. The sheath can have a tapered passageway or a passageway with a uniform diameter. The sheath preferably has a continuous taper along its outer surface from the proximal end to the distal end.

### TECHNICAL FIELD

This invention relates to a medical apparatus suitable for accessing a target site within the body of a patient, and more particularly, to a sheath suitable for use in introducing items like therapeutic agents or an interventional device into a bodily passageway of a patient.

### BACKGROUND

Introducer sheaths are in widespread use in the medical field for delivering a medical interventional device, such as a stent, to a target site within a bodily passageway of a patient, such as the vasculature. In order to reach the target site, the sheaths are often required to traverse tortuous pathways having sharp bends and angles. In some instances, and particularly when traversing such tortuous pathways, the sheaths exhibit a tendency to kink. Kinking reduces, and often collapses, the effective inner diameter of the sheath, thereby typically rendering the sheath unsuitable for its intended use.

The tendency of a sheath to kink is increased when the sheath is used to introduce an interventional device into one of the many smaller vessels that branch off from major vessels. In this event, the sheath may have insufficient flexibility at the very point where flexibility is most desired in order to enable proper positioning of the interventional device. In order to traverse the narrow confines of, e.g., the vascular system, the introducer sheath is typically formed of thin-wall construction. However, thin wall sheaths often have difficulty tracking narrow vessels, and exhibit an increased propensity to kink. Increasing the thickness of the sheath tube can minimally improve the level of kink resistance, as well as the trackability of the sheath. Any such increase in thickness, however, is inherently undesirable. The thickness increase limits the ability of the sheath to enter a narrow vessel, and reduces the diameter of the lumen when compared to the lumen of an otherwise similar thin-walled sheath. In addition, a larger diameter sheath necessitates the use of a larger entry opening than would otherwise be required or desirable.

One introducer sheath with improved kink resistance is disclosed in U.S. Pat. No. 5,380,304 to Parker. The introducer sheath described in the '304 patent comprises an inner liner formed of a lubricious fluoropolymer, such as polytetrafluoroethylene (PTFE). A coil is fitted around the inner PTFE liner, and an outer jacket formed of a heat-formable material, such as nylon or a polyether block amide, surrounds the inner liner and coil. The heat-formable material is heat shrunk onto the PTFE outer surface by enveloping it in a heat shrink tube, and heating the entire assembly until the material melts. As the heat-formable material melts, it flows between the spacings of the coil turns, and bonds to the outer diameter of the PTFE layer. The use of the coil in this device reinforces the tube of the sheath, and provides enhanced kink-resistance to an otherwise thin-walled introducer sheath.

The introducer sheath described in the '304 patent has proven to be particularly effective in delivering medical devices and medicaments to remote areas of a patient's vasculature without kinking. In order to minimize the cross-sectional profile (i.e., the outer diameter) of the sheath, the coil is generally formed of flat wire. By utilizing a flat wire coil, the sheath achieves a high level of kink resistance, and at the same time, maintains a low cross-sectional profile. The sheath described in the '304 patent enables the physician to routinely access, without kinking, target areas of the vasculature that had previously been difficult, or impossible, to reach. The '304 patent is incorporated herein by reference in its entirety.

With the continuous advances in the medical arts, more and more features have been developed to enhance the use of sheaths. For example, sheaths have been developed where only small sections of the sheaths are tapered in order to provide localized strain relief. However, the tapered sections often do not include reinforcement structures and thus are more susceptible to kinking. Some sheaths include an interwoven braid as the reinforcement structure, which is simply fitted to a mandrel by merely pulling the braid in tension longitudinally. However, sheaths with only an interwoven braid provide ineffective kink resistance, trackability, and crossability than sheaths with other reinforcement structures. Furthermore, during a procedure, a sheath is often left in place throughout the entire procedure, which provides possible sources of blood leakage around the sheath at the puncture site. Thus, what is needed is a sheath with an improved stiffness along its body length and with an improved kink resistance, as well as improved torqueability. What is also needed is a sheath sized and shaped to inhibit blood leakage around the sheath at the puncture site.

### BRIEF SUMMARY

In one embodiment, a flexible, kink-resistant sheath with a taper along at least a portion of its outer surface from the proximal end to the distal end is provided. The sheath is suitable for use in introducing items like therapeutic agents or an interventional device into a bodily passageway of a patient. The sheath includes an inner liner defining a passageway about a longitudinal axis that extends longitudinally through the passageway. A coil is fitted around at least a part of the inner liner. A portion of the coil has a series of windings that are locatable at a continuously smaller distance from the longitudinal axis to form a taper in a distal direction. The sheath further includes an outer layer positioned longitudinally over the coil, which is adapted to adhere to the inner liner. The tapered sheath with coil windings disposed to form a taper can provide a gradual transition in stiffness along its body length, as well as improved torqueability from the proximal region to the distal region of the sheath.

In other aspects of the sheath, the coil may be pre-wound to have spiral windings such that the windings are disposed at a continuously smaller radial distance from the longitudinal axis in a longitudinal direction. The sheath can have a tapered passageway or a passageway with a uniform diameter. The sheath can further include an intermediate layer positioned longitudinally in between the inner liner and the coil. The cross-section of the coil can be sized and shaped to include a surface substantially aligned with at least one of the passageway and the outer surface of the sheath.

In another embodiment, a method for forming a flexible, kink-resistant sheath having a taper along at least a portion of its outer surface from the proximal end to the distal end is provided. An inner polymer liner with a passageway extending therethrough is positioned around a mandrel. A coil is fitted around the inner polymer layer. The coil has a series of windings that are locatable at a continuously smaller distance from the longitudinal axis to form a taper in a distal direction. An outer polymer layer is positioned over at least a portion of the coil. An assembly comprising the mandrel, inner polymer layer, coil, and outer polymer layer is exposed to a sufficient amount of heat to at least partially melt the outer polymer layer such that a bond is formed between outer polymer layer and the inner polymer layer.

### BRIEF DESCRIPTION OF THE DRAWINGS

FIG. 1 is a side elevation view of a flexible, kink-resistant sheath, shown in combination with a dilator and a hub.

FIG. 2 is a longitudinal cross-sectional view of a portion of the wall of the sheath of FIG. 1, taken along line 2-2, depicting a tapered passageway.

FIG. 2A is a longitudinal cross-sectional view similar to FIG. 2 of a portion of the wall of another embodiment of a sheath, depicting a passageway with a uniform cross-section.

FIG. 3A is close-up, detailed partial view of the wall of a sheath, depicting a cross-section of a coil.

FIG. 3B is close-up, detailed partial view of the wall of a sheath, depicting a cross-section of a coil.

FIG. 3C is close-up, detailed partial view of the wall of a sheath, depicting a cross-section of a coil.

FIG. 3D is close-up, detailed partial view of the wall of a sheath, depicting a cross-section of a coil.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the inventive sheath, as well as the axial ends of various component features. The term “proximal” is used in its conventional sense to refer to the end of the apparatus (or component thereof) that is closest to the operator during use of the apparatus. The term “distal” is used in its conventional sense to refer to the end of the apparatus (or component thereof) that is initially inserted into the patient, or that is closest to the patient during use.

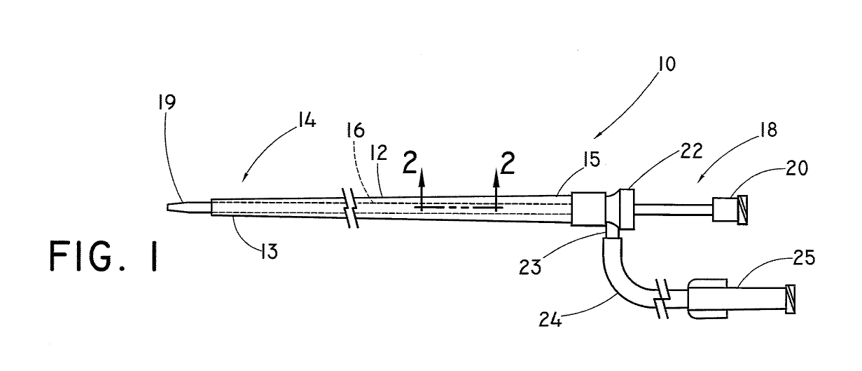

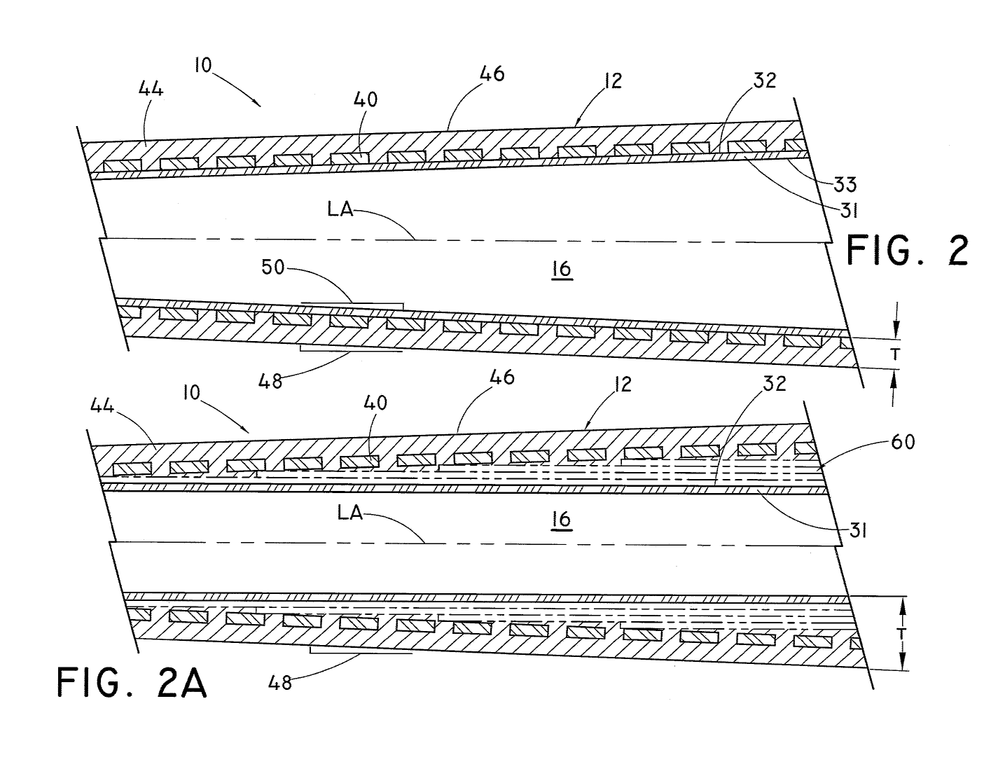

FIG. 1 shows an exemplary flexible introducer sheath 10 having a taper with an improved stiffness along at least a portion of its body length and with an improved kink resistance so that trackability of said sheath through tortuous pathways of the bodily passageway of the patient is improved. Sheath 10 can be suitable for use in introducing items like therapeutic agents or an interventional device into a bodily passageway of a patient. Sheath 10 includes a tube 12, having a distal portion 13 and a proximal portion 15. Preferably, distal portion 13 also includes a distal end 14 being tapered in a similar fashion as the body of the tube, or may even be tapered to a different degree. An inner passageway 16 extends through sheath 10, as shown in FIG. 2.

According to FIG. 1, sheath 10 can be used in combination with an optional dilator 18 and a connector hub 22. Dilators and connector hubs for use with introducer devices, such as sheath 10, are well known, and the particular dilator and hub illustrated in FIG. 1 may be replaced with various other dilators and hubs known in the art. As shown herein, dilator 18 extends longitudinally through the passageway of the sheath. The dilator includes a tapered distal end 19 for accessing and dilating a vascular access site, e.g., over a wire guide (not shown) by any conventional vascular access technique, such as the well-known Seldinger technique. A Luer lock connector 20 may be attached at the proximal end of the dilator for connection to a syringe or other medical apparatus in well known fashion.

Connector hub 22 is attached about the proximal end of the sheath during use. Connector hub 22 may include one or more conventional silicone disks (not shown) for preventing the backflow of fluids therethrough. Connector hub 22 may also include a side arm 23, to which a polymeric tube 24 and a conventional connector 25 may be connected for introducing and aspirating fluids therethrough in conventional fashion.

FIG. 2 is a longitudinal cross-sectional view of a portion of the tube 12 of sheath 10 of FIG. 1, which illustrates the layered structure of the sheath. The views of sheath 10 in FIGS. 2 and 2A do not include the optional dilator 18. As illustrated, tube 12 comprises a liner 31, having a radially outer surface 32. A reinforcing member, such as coil 40, is wound or otherwise fitted around the radially outer surface 32 of liner 31. A polymeric outer layer or jacket 44 is also provided around the coil for adhering to the outer surface 32 of liner 31 through the spaced windings of the coil 40.

As stated earlier, sheath 10 has a taper along at least a portion of the outer surface 46 of the tube 12, if not along its entire outer surface 46. The tapering rate 48 of the outer surface of the tube 12 is suitable to improve stiffness along its body length and/or match the tapering of the body vessel. The tapering rate 48 can be, e.g., approximately 0.1 mm to about 0.6 mm per 10 cm in length. One of the advantages of a continuous tapered tube for a sheath is the gradual change in stiffness from the distal end to the proximal end as there are no discrete stiffness sections. Another advantage is a tapered sheath can provide a snug fit through the puncture site to minimize blood leakage. Even if the fit between the sheath and the puncture site becomes loose enough to allow blood leakage, the bleeding can be stopped and the procedure continued simply by advancing the sheath a suitable distance in the puncture site to restore the snug fit. A gradual taper may help also in transitioning to a bifurcated body vessel where conventional sheaths have sharper transitions that tend to catch at the transition, and provide a better fit to the vessel at the distal end.

Liner 31 is typically formed of a lubricious material. Preferably, the lubricious material comprises a fluoropolymer, such as PTFE or FEP.

Lubricious liners for sheaths are well known in the medical arts, and those skilled in the art can readily select an appropriate liner for a particular use. The lubricious material provides a slippery, low friction radially inner surface 33 to ease insertion and/or withdrawal through passageway 16 of the dilator or medical interventional device, such as a stent. The radially outer surface 32 of liner 31 may be roughened in any conventional manner, such as by machine grinding or chemical etching, to form irregularities on the surface to facilitate bonding with coil 40 and/or outer layer 44. The wall of the liner will also preferably have sufficient structural integrity to prevent the coil turns from protruding into inner passageway 16.

Outer layer 44 may generally be formed from any composition commonly used for such purposes in a medical device. Non-limiting examples of such composition include a polyether block amide, nylon, polyurethane or the like. Other outer layer compositions that are capable of securely bonding, adhering, or otherwise securely engaging the liner and/or the coil may be substituted. It is preferred to form outer layer 44 from a material having a lower melt temperature than that of liner 31. To this end, the tube can be heated at a temperature suitable to melt the outer layer.

Coil 40 may be formed from well-known materials for such use in the medical arts, such as a metal, a metal alloy (e.g., stainless steel or a shape memory composition such as nitinol), a multi-filar material, or a composite material. In order to minimize the cross-sectional profile (i.e., outer diameter) of the sheath, it is preferred to provide a coil with a low profile, such as a conventional flat wire construction. However, those skilled in the art will appreciate that coil materials of other cross-sectional configurations, such as round, oval, and various other geometric configurations like the ones described herein, may be substituted.

Besides the tapering outer surface of the sheath, FIG. 2 illustrates that sheath 10 includes a passageway 16 having a taper or an increasingly smaller cross-sectional area in the distal direction. One of the advantages of a tapered passageway is that it can allow for improved flow rate therethrough for fluid applications such as the delivery of contrast media or therapeutic agents, and for embolic applications such as delivery of microspheres that otherwise may tend to clog the passageway especially at kinkable regions of the tube. Another advantage can be found in the delivery of medical devices such as catheters or embolic devices, where there is less friction due to the gradual taper of the passageway.

The tapering rate 50 of passageway 16 may be approximately the same as the tapering rate 48 of the outer surface 46 of tube 12, and may be even such that the wall thickness “t” of the tube is maintained substantially uniform. For example, the tapering rate 50 can be, e.g., approximately 0.1 mm to about 0.6 mm per 10 cm in length, where the wall thickness is approximately 0.015 inches to 0.03 inches. Alternatively, the tapering rates of the passageway and the outer surface of the tube may be different when balancing the needs for flexibility and flow rate so that the wall thickness becomes increasingly smaller in the distal direction. For example, the tapering rate 50 of the passageway can be, e.g., approximately 0.1 to about 0.2 mm per 10 cm in length and the tapering rate 48 of the outer surface can be greater, e.g., approximately 0.5 to about 0.6 mm per 10 cm in length.

In some embodiments, coil 40 has windings having a constant diameter in a longitudinal direction. The constant diameter wound coil can then be applied over a tapered mandrel with liner 31 already disposed thereon so that the windings are located at a continuously smaller distance from the longitudinal axis LA in a distal direction. For instance, the diameter of the coil can be approximately at least the same size as the diameter of the smaller end of the tapered mandrel so that one end of coil 40 can contact liner 31 proximate the large end, while the other end is wrapped around the liner along the tapering mandrel for a radially compressed fit. Those skilled in the art are aware that coil 40 can be approximately at least the same size as the diameter of the large end of the tapered mandrel so that one end of the coil can contact the liner proximate the large end while the other end is then wrapped around the liner along the tapering surface for a radially expanded fit. Adhesives, such as cyanoacrylate, can be used to attach more securely portions of the coil to the liner.

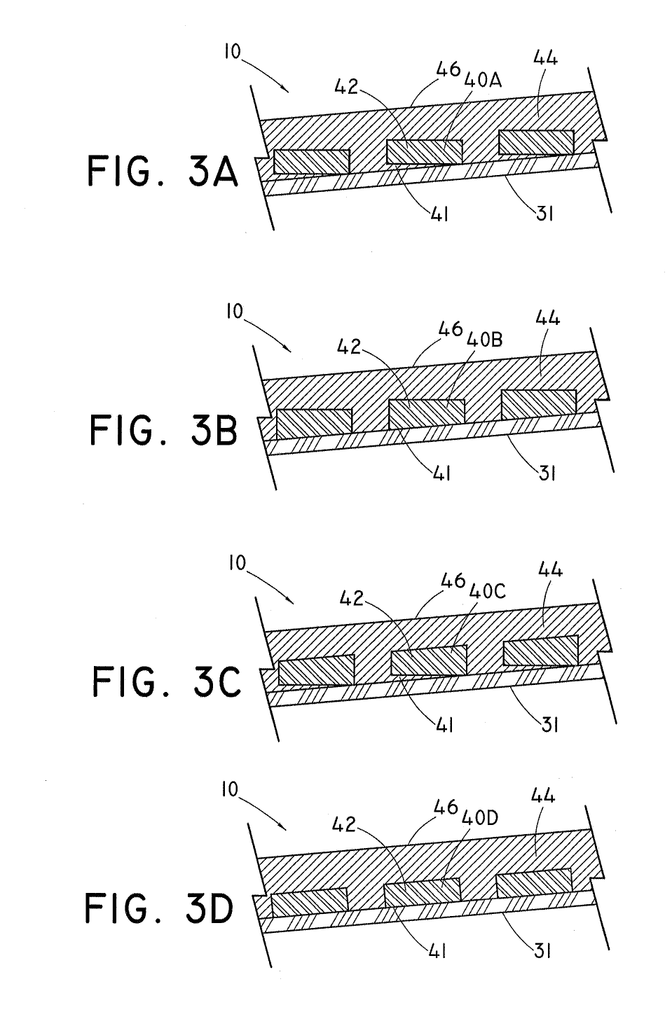

It can be also appreciated that during heating of tube 12, the coil, referred to as 40A having a rectangular cross-section, may not be fully aligned with the outer surface 46 and/or the passageway 16 of the tube 12, as shown in FIG. 3A. This is due to the resiliency of the windings of coil 40A and their capability to return to a natural configuration of a constant diameter wound coil, which can cause each winding to slightly orient itself away from the tapered surfaces. This allows the edge or surface of the coil to extend radially inward and/or outward such that ridges can be formed along the body of the tube, potentially causing a reduction in kink resistance. A ridged tube along the outside makes pushability of the tube along the body vessel wall more difficult due to the increase in friction from a ridged outer surface. A ridged tube along the inside makes pushability of insertable medical devices more difficult due to the increased in friction from a ridged inner surface.

To overcome this tendency of the constant diameter wound coil, the cross-section of the coil can be ground or otherwise modified so that at least one of the inner surface 41 and the outer surface 42 of coil 40, if not both, aligns with the taper of the outer surface 46 of the tube 12 and/or passageway 16. As appreciated by those skilled in the art, the cross-section of the coil can be formed from the onset or modified by a grinding and/or a drawing process, as well as other techniques known in the art.

FIGS. 3B-3D show various cross-sections of the coil 40 having improved alignability to avoid ridge formation. FIG. 3B depicts the inner surface 41 of the winding of the coil, referred to as 40B, being ground or otherwise shaped (more wedge-like) to align with the tapered inner passageway 16. This embodiment can reduce the likelihood of ridge formation along the inner surface of the passageway. FIG. 3C depicts the outer surface 42, instead of the inner surface 41, of the winding of the coil, referred to as 40C, being ground or otherwise shaped (more wedge-like) to align with the tapered outer surface 46 of the tube 12. This embodiment can reduce the likelihood of ridge formation along the outer surface of the tube. FIG. 3D depicts both the inner and outer surfaces, 41, 42 of the winding of the coil, referred to as 40D, being ground or otherwise shaped (similar to a parallelogram) to align with both the tapered inner passageway 16 and with the tapered outer surface 46 of the tube 12, respectively. This embodiment can reduce the likelihood of ridge formation along the inner surface of the passageway and along the outer surface of the tube.

In other embodiments, the windings of the coil 40 can be wound to have an increasingly larger or smaller diameter in a longitudinal direction. In other words, the coil windings are wound at a continuously smaller distance in a distal direction to form a spirally wound coil such that the windings of the coil are located at a continuously smaller distance from the longitudinal axis LA. To this end, including a spirally wound coil can take advantage of the resiliency of windings and their capability to return to a natural configuration of a spiral with an increasingly smaller diameter in the distal direction. Preferably, the tapering rate of the spirally wound coil is approximately the same as the tapering rate of the outer surface 46 of the tube 12, taking into consideration the cross-section of the coil, the general width and thickness of the coil, the spacing between adjacent windings, and the like.

The spirally wound coil can then be applied over a tapered mandrel with liner 31 already disposed thereon. For instance, the spirally wound coil can be placed on a relatively larger cross-section of the tapered mandrel having approximately the same tapering rate as the spirally wound coil so that one end of the coil can be attached to the liner proximate the lager cross-section. The other end of the spirally wound coil is then wrapped around the liner along the tapered surface of the mandrel for a radially compressed fit. Those skilled in the art are aware that the spirally wound coil can be placed on a relatively smaller section of the tapered mandrel having approximately the same tapering rate as the spirally wound coil so that one end can be attached to the liner proximate the smaller cross-section. The other end of the spirally wound coil is then wrapped around the liner along the tapering surface of the mandrel for a radially expanded fit. Similar to the modified cross-sections depicted in FIGS. 3A-3D, the cross-section of the spirally wound coil can be ground or otherwise modified so that at least one of the inner surface 41 and the outer surface 42 of coil 40, if not both, aligns with the taper of the outer surface 46 and/or passageway 16 of the tube 12.

FIG. 2A is a longitudinal cross-sectional view similar to FIG. 2 of a portion of the tube 12 having an alternative construction. FIG. 2A shows the tube 12 of sheath 10 having liner 31 with a substantially uniform inner diameter extending the entire length of passageway 16. One advantage of a uniform passageway is that it can allow the passage of an interventional device having the largest possible diameter therethrough. In this instance, the inner diameter of the passageway can be, e.g., approximately 0.065 inches to 0.165 inches. Since the outer surface 46 of tube 12 tapers at tapering rate 48 and the passageway has a uniform inner diameter, the wall thickness of the tube becomes increasingly smaller in the distal direction. The varied thickness along the entire length of the tube 12 can improve flexibility by distributing bending forces along the entire sheath.

An intermediate layer 60 can be used to dispose the coil 40 to align with the tapered outer surface 46 of the tube 12. The intermediate layer can be applied along the entire length of the tube. Alternatively, the intermediate layer can be applied to an intermediate portion of the tube where a distal portion of the tube extending distally past the intermediate portion has a constant cross-section, rather than being tapered. In this configuration, ridge formation along the inner surface of the passageway due to the coil will be less likely. Intermediate layer 60 is disposed between coil 40 and liner 31. Intermediate layer 60 has a radially inner surface 62 that substantially aligns with the passageway 16 having the uniform diameter and a radially outward surface 64 that substantially aligns with the tapering outer surface 46 of the tube 12.

Intermediate layer 60 may be formed of the same material as outer layer 44 in order for the layers to thermally bond to one another more easily. To prevent the coil from returning to its natural state, i.e., penetrating through the outer surface 64 of intermediate layer 60, the intermediate layer may be formed of the same material as outer layer 44 with a different durometer. Optionally, the intermediate layer 60 may even be formed of a different material that is still capable of bonding with the outer layer 44. Different durometer materials along different sections of the tube can vary the stiffness of the tube. For example, outer layer 44 can comprise a material having a high durometer, such as a durometer between about 60 and 80 on the Shore D scale, as such high durometer materials provide favorable kink resistance to the tube, and also provide sufficient strength to enable the tube to be guided through small diameter passageways in the vasculature. Intermediate layer 60 can include a material having a durometer of about 30 to 60 on the Shore D hardness scale. Such materials, e.g., a nylon elastomer, more preferably have a durometer of about 35 to 50, and most preferably about 40.

Intermediate layer 60 can comprise several tube sections positioned with respect to one another so as to form the gradually thicker intermediate layer shown in FIG. 2A. In this instance, multiple coaxial layers (shown in dashed lines) of increasingly smaller lengths are disposed around the structure comprising the mandrel and inner liner so that distal ends of the coaxial layers terminate longitudinally along the structure at different locations. It can be appreciated that varying the thickness of the coaxial layers and/or the lengths of the coaxial layers can vary the flexibility of the sheath. Optionally, the intermediate layer can be formed of a material with the tapering wall thickness show in FIG. 2A. Possible other materials for the intermediate layer include thermoplastic polymers, such as polyethylene, polyurethane, polyether block amide, nylon or the like. Instead of being applied between the coil and the inner liner, the intermediate layer can be applied between the coil and the outer layer or even outside the outer layer to form a tapering outer surface of the tube. In this example, the coil may not be locatable at a continuously smaller distance from the longitudinal axis LA to form a taper in a distal direction, but may be disposed at a substantially uniform distance from the longitudinal axis LA.

A method of forming the tube 12 of sheath 10 will now be described. Initially, the liner 31 is positioned over a supporting tapered mandrel in well-known fashion for the sheath embodiment with a tapered passage shown in FIG. 2. The liner can be pre-formed with a funnel shape having a similar tapering rate as the mandrel so that the liner can fit snugly along the mandrel. Alternatively, the liner can be formed of tubing of various diameters and aligned along the tapered mandrel from smallest to largest in attempt to match the tapering rate of the mandrel.

The coil 40 may be wrapped, wound, compression fitted, or otherwise applied around the outer surface 32 of liner 31 in a conventional fashion, regardless if the coil is a constant diameter wound coil or a spirally wound coil as described above. Techniques for applying a coil to a substrate in a sheath are now well known, and various conventional techniques will be suitable for use herein. Non-limiting examples of such techniques are described in the incorporated-by-reference citation.

Outer layer 44 is then applied to the outer surface of the liner to sandwich the coil. Generally speaking, any conventional technique for engaging the outer layer 44 with the liner and/or the coil may be utilized. In one preferred technique, outer layer 44 comprises a sleeve formed of a composition that has a lower melt temperature than that of the material of the liner 31. Those skilled in the art will appreciate, however, that virtually any composition that is capable of forming a secure bond with the liner and/or coil may be utilized. The sleeve is positioned over the structure comprising the coil, the liner and the mandrel. The outer layer sleeve can be pre-formed with a funnel shape having a similar tapering rate as the mandrel. Alternatively, the outer layer sleeve can be formed of tubing of various diameters and aligned along the tapered mandrel from smallest to largest in attempt to match the tapering rate of the mandrel.

For the sheath embodiment having a uniform diameter passageway, shown in FIG. 2A, the liner 31 is initially positioned over a supporting mandrel with a uniform diameter in well-known fashion. The coil 40 may be wrapped, wound, compression fitted, or otherwise applied around the outer surface 32 of liner 31 and/or the outer surface of the 64 of intermediate layer 60 in a conventional fashion, regardless if the coil is a constant diameter wound coil or a spirally wound coil as described above. Outer layer 44 is then applied to the outer surface of the liner and/or the intermediate layer to sandwich the coil. It can be appreciated that the intermediate layer 60 can be applied to the embodiment of FIG. 2 for suitably varying the tapering rates of the outer surface of sheath and the passageway individually.

For all of the embodiments described herein, the entire assembly (comprising at least one of the outer sleeve, coil, intermediate layer, liner and mandrel) is then placed in a heat shrink enclosure formed of a material commonly utilized for such purposes, such as fluorinated ethylene propylene (FEP). The heat shrink enclosure can be pre-formed with a funnel shape having a similar tapering rate as the mandrel to fit along the assembly. Alternatively, the heat shrink enclosure can be formed of tubing of various diameters and aligned along the tapered mandrel from smallest to largest to attempt to match the tapering rate of the mandrel. The heat shrink enclosure enclosing the assembly is then placed in an oven, and heated to a temperature (e.g., 400-500° F. (204-260° C.)) sufficient to at least partially melt the outer layer composition. The melted compositions flow between the turns of the coil, resulting in the formation of a secure bond between the outer surface of the liner and/or coil and the outer layer composition, and intermediate layer if included. Following formation of the bonds as described above, the assembly is allowed to cool, and thereafter removed from the heat shrink enclosure. The mandrel is then removed from the inner liner.

The tube described hereinabove preferably utilizes a coil reinforcement instead of a braid reinforcement for improved kink resistance. The windings of the coil reinforcement are locatable at a continuously smaller distance from a longitudinal axis to form a taper in a distal direction for improved torqueability of the tube gained from the mechanical advantage at the proximal end relative to the distal end. The torqueability may be suitable to avoid the addition of a braid reinforcement, which can result in a thinner walled sheath. A tapered outer surface sheath with a tapered coil reinforcement thus allows for better control at the distal end for the clinician and improved torqueability, kink resistance, and flexibility, making the sheath more trackable through tortuous anatomy.

Although the tube described hereinabove preferably utilizes a coil reinforcement, the teachings of the present invention are also applicable to tubes or other devices having other structures disposed therewithin. For example, in some embodiments, a braided reinforcement formed of interwoven wires may be used in addition to the coil reinforcement. Those skilled in the art will appreciate that all dimensions, compositions, etc., described herein are exemplary only, and that other appropriate dimensions, compositions, etc., may be substituted in an appropriate case. For example, the respective thicknesses of the inner liner and the outer layer for a sheath are conventional, and may be varied based upon the intended use of the tube. If desired, the tube can be formed to have one or more segments of varying durometer along its length, typically aligned in a sequence of decreasing durometer from the proximal end to the distal end in well-known fashion. Additionally, other features commonly found in tubes, such as radiopaque markers, rings, coatings, etc., may also be incorporated into the inventive structure in well-known manner.

Although the foregoing detailed description focuses on tubes being tapered along the entire outer surface and/or or along the entire inner passageway, it is appreciated that persons skilled in the art can taper the outer surface of the tube and/or passageway along only a portion of the length of the tube and/or passageway with the teaching of the detailed description.

Drawings in the figures illustrating various embodiments are not necessarily to scale. Some drawings may have certain details magnified for emphasis, and any different numbers or proportions of parts should not be read as limiting, unless so-designated in the present disclosure. Those skilled in the art will appreciate that embodiments not expressly illustrated herein may be practiced within the scope of the present invention, including those features described herein for different embodiments may be combined with each other and/or with currently-known or future-developed technologies while remaining within the scope of the claims presented here. It is therefore intended that the foregoing detailed description be regarded as illustrative rather than limiting. And, it should be understood that the following claims, including all equivalents, are intended to define the spirit and scope of this invention

1. A sheath having a proximal end and a distal end, the sheath comprising:
an inner liner defining a passageway about a longitudinal axis extending longitudinally therethrough;
a coil fitted around at least a part of the inner liner, the coil having a series of windings, wherein a portion of said windings of the coil are locatable at a continuously smaller distance from the longitudinal axis to form a taper in a distal direction; and
an outer layer positioned longitudinally over said coil to adhere to the inner liner such that at least a portion of an outer surface of said sheath has a taper.

2. The sheath of claim 1, wherein said coil is spirally wound.

3. The sheath of claim 2, wherein at least a portion of said passageway has a taper.

4. The sheath of claim 3, wherein the taper of said passageway is tapered at a rate approximately equal to a rate of the taper of the outer surface of said sheath.

5. The sheath of claim 3, wherein the taper of said passageway is tapered at a rate different than a rate of the taper of the outer surface of said sheath.

6. The sheath of claim 1, wherein the outer surface of said sheath is tapered from the proximal end to the distal end of said sheath.

7. The sheath of claim 1, further comprising an intermediate layer positioned longitudinally in between said inner liner and said coil, the intermediate layer shaped to dispose the coil at the continuously smaller distance from the longitudinal axis to form the taper in the distal direction, wherein the passageway has a substantially uniform cross-section.

8. The sheath of claim 7, wherein said intermediate layer has a continuously larger thickness in the proximal direction.

9. The sheath of claim 8, wherein the continuously larger thickness of said intermediate layer is at a rate approximately equal to a rate of the taper of the outer surface of said sheath.

10. The sheath of claim 1, wherein the coil windings have outer and inner surfaces, at least one of the surfaces is substantially aligned with at least one of the passageway and the outer surface of the sheath.

11. The sheath of claim 10, wherein the outer surface is in substantial alignment with the outer surface of the sheath and the inner surface is in substantial alignment with the passageway.

12. A sheath having a proximal end and a distal end, the sheath comprising:
an inner liner defining a passageway about a longitudinal axis extending longitudinally therethrough;
a coil fitted around at least a part of the inner liner, the coil having a series of windings, wherein said windings of the coil are spirally wound at a continuously smaller distance from the longitudinal axis to form a taper in a distal direction; and
an outer layer positioned longitudinally over said coil to adhere to the inner liner such that an outer surface of said sheath has a continuous taper from the proximal end to the distal end thereof.

13. The sheath of claim 12, wherein said passageway has a taper from the proximal end to the distal end at a rate approximately equal to a rate of the taper of the outer surface of said sheath.

14. The sheath of claim 12, wherein said passageway has a taper from the proximal end to the distal end at a rate different than a rate of the taper of the outer surface of said sheath.

15. The sheath of claim 12, wherein said passageway has a substantially uniform cross-section.

16. The sheath of claim 15, further comprising an intermediate layer positioned longitudinally in between said inner liner and said coil to adhere to the inner liner and the outer layer.

17. The sheath of claim 1, wherein said coil is spirally wound at a rate of about 0.1 mm to about 0.6 mm per 10 cm length.

18. A method for forming a tapered sheath, comprising:
providing an inner polymer liner, said inner liner having a passageway extending therethrough, and having an outer surface;
positioning said inner liner around a mandrel;
positioning a coil around the inner polymer layer, said coil having a series of windings, wherein said windings of the coil are locatable at a continuously smaller distance from the longitudinal axis to form a taper in a distal direction;
applying an outer polymer layer over at least a portion of said coil; and
exposing an assembly comprising the mandrel, inner polymer layer, coil and outer polymer layer to a sufficient amount of heat to at least partially melt the outer polymer layer such that a bond is formed between outer polymer layer and the inner polymer layer, and such that said sheath has a continuous taper from the proximal end to the distal end thereof.

19. The method of claim 18, wherein said step of positioning said inner liner around a mandrel further comprises positioning said inner liner around a tapered mandrel such that said passageway is tapered.

20. The method of claim 18, wherein further comprising applying an intermediate layer between said inner polymer liner and said coil.
